(19) United States
(12) Patent Application Publication
(10) Pub. No.: US 2023/0184775 A1
(43) Pub. Date: Jun. 15, 2023

(54) KINDLIN-1 AS A MARKER OF SENSITIVITY TO EGFR/RAS PATHWAY INHIBITORS

(71) Applicant: INSTITUT CURIE, Paris (FR)

(72) Inventors: Keltouma Driouch, Paris (FR); Rosette Lidereau, Paris (FR)

(73) Assignee: INSTITUT CURIE, Paris (FR)

(21) Appl. No.: 17/925,008

(22) PCT Filed: May 11, 2021
(86) PCT No.: PCT/EP2021/062451

§ 371 Date: Nov. 13, 2022

(30) Foreign Application Priority Data
May 12, 2020 (EP) 20305484.6

(51) Int. Cl. G01N 33/574; G01N 33/68

(52) U.S. Cl. CPC G01N 33/57484; G01N 33/6872; G01N 2333/485

(57) ABSTRACT

The present invention concerns an ex vivo method for selecting patient having cancer as eligible to EGFR/RAS pathway inhibitor therapy, an EGFR/RAS pathway inhibitor for use for treating cancer in a patient, and a method for prognosis cancer outcome or progression.

The present invention concerns an ex vivo method for selecting patient having cancer as eligible to EGFR/RAS pathway inhibitor therapy, an EGFR/RAS pathway inhibitor for use for treating cancer in a patient, and a method for prognosis cancer outcome or progression.

Cancer is the second leading cause of death globally, and is responsible for an estimated 9.6 million deaths in 2018. Globally, about 1 in 6 deaths is due to cancer. The most common types of cancer in males are lung cancer, prostate cancer, colorectal cancer and stomach cancer. In females, the most common types are breast cancer, colorectal cancer, lung cancer and cervical cancer. A correct cancer diagnosis is essential for adequate and effective treatment because cancer requires a specific treatment regimen that encompasses one or more modalities such as surgery, radiotherapy, and chemotherapy. Some treatments having side effects, it is preferable to have way to distinct sensitive patient on which the treatment will have important positive effect to the resistant patient.

The epidermal growth factor receptor (EGFR) is a tyrosine kinase receptor and is known as a proto-oncogene. The superfamily of Rat Sarcoma protein (RAS) is a family of small proteins with GTPase activity. It is made up of more than 150 members divided into five main families: RAS, RHO, RAB, ARF and RAN. RAS is activated by the Sos/Grb2 complex following activation of EGFR. Several treatments of cancer are EGFR/RAS pathway inhibitors.

There is a need for new biological parameters allowing distinguishing patients who will subsequently benefit from EGFR/RAS pathway inhibitor therapy from those who are non-responders, and thus avoiding them unnecessary treatments.

Kindlin-1 is a focal adhesion protein involved in the activation of β-integrins and therefore participate in important cellular processes such as cell adhesion, proliferation or migration. Altered expression levels of this protein have been reported in a broad range of cancers.

The inventors have shown that, at a clinical level, Kindlin-1 expression is higher in the tumors than in the normal tissues in different cancer types (breast, lung, colon, bladder...) and its up-regulation is associated with a worse prognosis. At an experimental level, the role of Kindlin-1 in tumor invasion and metastasis was validated by combining in vitro and in vivo models. Kindlin-1 expression was found to increase cellular proliferation, migration and invasion. Finally, it was shown that Kindlin-1 depletion in a breast cancer mouse model inhibits primary tumor growth and avoids the development of lung metastases.

### SUMMARY OF THE INVENTION

Surprisingly, the inventors have discovered that Kindlin-1 expression is associated with the activation of EGFR/RAS pathway in several solid cancers, including breast, lung, pancreas, bladder and head and neck cancers. Kindlin-1 expression permits to identify EGFR/RAS-driven cancer patients with worst survival outcome. Kindlin-1 expression is also associated with sensitivity to EGFR/RAS pathway inhibition in a broad range of epithelial cancers, and in breast cancer.

Thus, in a first aspect, the invention provides a method, preferably an ex vivo method, for selecting a patient having cancer as eligible to EGFR/RAS pathway inhibitor therapy, comprising:
a) Measuring Kindlin-1 expression level in cancer cells of said patient having cancer;
b) Selecting the patient as eligible to EGFR/RAS pathway inhibitor therapy if the measured Kindlin-1 expression level in cancer cells of said patient having cancer is equal to, or above, a reference level.

Also provided is an EGFR/RAS pathway inhibitor for use for treating cancer in a patient, wherein said patient has been identified has having a Kindlin-1 expression level in cancer cells equal to, or above, a reference level.

In a further aspect, the invention provides a method for prognosis cancer outcome or progression in a patient suffering from EGFR/RAS-driven cancer, comprising:
a) Measuring Kindlin-1 expression level in cancer cells of said patient suffering from EGFR/RAS-driven cancer;
b) Determining that the cancer is likely to progress or have a poor outcome if the measured Kindlin-1 expression level in cancer cells of the patient is equal to, or above, a first reference level; or determining that the cancer is unlikely to progress or have a poor outcome if the measured Kindlin-1 expression level in cancer cells of the patient is below a second reference level.

The invention also concerns an EGFR/RAS pathway inhibitor or a Kindlin-1 inhibitor for use for treating cancer in a patient suffering from EGFR/RAS-driven cancer, wherein said patient has been identified as having a cancer likely to progress or having a poor outcome, preferably said patient has been identified according to the method of prognosis cancer outcome or progression of the invention.

### DESCRIPTION OF THE INVENTION

The invention provides a method, for selecting a patient having cancer as eligible to EGFR/RAS pathway inhibitor therapy, comprising:
a) Measuring Kindlin-1 expression level in cancer cells of said patient having cancer;
b) Selecting the patient as eligible to EGFR/RAS pathway inhibitor therapy if the measured Kindlin-1 expression level in cancer cells of said patient having cancer is equal to, or above, a reference level.

Preferably the method of the invention is an ex vivo method. Preferably, cancer cells taken from the patient are used.

### EGFR/RAS Pathway Inhibitor Therapy

The term “EGFR/RAS pathway” refers to all the reactions and metabolites, which are produced by the activation of EGFR and/or the activation of RAS and the following sequence of reactions and metabolites. It includes in particular the RAS-RAF-MEK½-ERK½ signaling axis, i.e RAS proteins (K-RAS, H-RAS, N-RAS), BRAF, MEK (MEK½), and MAPK (ERK½) proteins. The term “EGFR/RAS pathway” also includes the reactions required for the activation of EGFR and/or RAS.

“Therapy” or “treatment” includes reducing, alleviating, inhibiting, or eliminating the causes of a disease or pathological conditions, as well as treatment intended to reduce, alleviate, inhibit or eliminate symptoms of said disease or pathological condition. In particular herein these terms includes reducing the tumor size, slowing the tumor growth, eliminating the tumor, and/or inhibiting the apparition of metastasis.

By “EGFR/RAS pathway inhibitor therapy”, it is meant a therapy using the administration of at least one EGFR/RAS pathway inhibitor. Such inhibitor can be a direct inhibitor of EGFR/RAS activation or an inhibitor of a protein in the EGFR/RAS signaling axis. The EGFR/RAS pathway inhibitor is in particular an EGFR inhibitor, a RAS inhibitor, a BRAF inhibitor, a MEK inhibitor or a MAPK inhibitor.

“EGFR inhibitor” includes all inhibitors of EGFR known to the skilled in the art. In particular, EGFR inhibitor includes anti-EGFR antibodies and small molecule inhibitors. Anti-EGFR antibodies include Cetuximab, Panitumumab, Zalutumumab, Nimotuzumab, and Matuzumab. Preferably, the anti-EGFR antibody is Cetuximab or Panitumumab. Small molecule inhibitors of EGFR include Erlotinib, Dabrafenib, Gefitinib, Osimertinib, Pelitinib, AZD3759 (CAS number: 1626387-80-1), afatinib, brigatinib, icotinib, Dacomitinib (PF-00299804, Vizimpro), Avitinib (AC0010MA), Olmutinib (HM61713) and lapatinib. Preferably, a small molecule inhibitor of EGFR is selected from the group consisting of Erlotinib, Dabrafenib, Gefitinib, Osimertinib, Pelitinib and AZD3759 (CAS number: 1626387-80-1). More preferably, a small molecule inhibitor of EGFR is selected from the group consisting of Erlotinib, Gefitinib, Osimertinib, Pelitinib and AZD3759 (CAS number: 1626387-80-1).

“MEK inhibitor” includes all inhibitors of MEK½ known to the skilled in the art. In particular, the MEK inhibitor is selected from the group consisting of PD0325901 (CAS Number: 391210-10-9), CI-1040 (CAS Number: 212631-79-3), Refametinib, Selumetinib, Trametinib, Cobimetinib, GDC-0623 (RG 7421) and TAK-733 (CAS Number: 1035555-63-5). Preferably, the MEK inhibitor is selected from the group consisting of PD0325901, CI-1040, Selumetinib, Refametinib and Trametinib.

“BRAF inhibitor” includes all inhibitors of BRAF known to the skilled in the art. In particular, the BRAF inhibitor is selected from the group consisting of Vemurafenib ((PLX4032, Plexxikon; RG7204, R05185426), PLX-4720 (CAS Number: 918505-84-7), Dabrafenib (GSK21 18436), Sorafenib (BAY43-9006), GDC-0879 (CAS Number: 905281-76-7), PLX4720 (CAS Number: 918505-84-7), BMS-908662 (XL281, CAS Number: 870603-16-0), LGX818 (CAS Number: 1269440-17-6), PLX3603 (RO5212054), ARQ-736 (CAS number: 1228237-57-7), DP-4978 (CAS number: 1454682-72-4) and RAF265 (CAS number: 927880-90-8). Preferably, the BRAF inhibitor is selected from the group consisting of PLX-4720 (CAS Number: 918505-84-7) and Dabrafenib (GSK21 18436).

“RAS inhibitor” includes all inhibitors of RAS, KRAS, HRAS, NRAS known to the skilled in the art. In particular, the RAS inhibitor is AMG510 (CAS Number: 2252403-56-6), SCH-54292 (CAS Number: 188480-51-5), SCH53239 (CAS Number: 188480-49-1), TLN-4601 (CAS Number: 733035-26-2), Salirasib, Deltarasin, Lonafarnib, Tipifarnib, L-778,123 (CAS Number: 253863-00-2), SML-8-73-1, SML-10-70-1 (CAS Number: 1536470-98-0), MRTX849 (CAS Number: 2326521-71-3), AZD4785, ARS-1620 (CAS Number: 1698055-85-4), KRAS inhibitor 12 (CAS number: 1469337-95-8). Preferably, the RAS inhibitor is AMG510 (CAS Number: 2252403-56-6) or KRAS inhibitor 12 (CAS number: 1469337-95-8).

“MAPK inhibitor” or “ERK½ inhibitor” includes all inhibitors of ERK½ known to the skilled in the art. In particular, the ERK½ inhibitor is selected from the group consisting of Ulixertinib, SCH772984 (CAS number: 942183-80-4), Ravoxertinib (GDC-0994) and LY3214996 (CAS Number: 1951483-29-6).

### Kindlin-1 Expression Level

By “Kindlin-1 expression level”, it is meant the level of expression of messenger RNA (mRNA) encoding Kindlin-1 and/or the level of expression of the Kindlin-1 protein.

Preferentially, the level of expression of Kindlin-1 protein is measured using a specific ligand of Kindlin-1 , such as for example an antibody, preferably monoclonal antibody, a Fab fragment, an scFv or a nanobody, specific for Kindlin-1. The level of expression may then be measured by means of any method known to those skilled in the art, such as for example by means of a Western Blot or an ELISA test. The level of expression of Kindlin-1 protein may also be measured by mass analysis, such as mass spectrometry. Qualitative and quantitative mass spectrometric techniques are known and used in the art. A quantitative LC-MS/MS can also be used.

Preferentially, the level of expression of mRNA of the gene encoding Kindlin-1 is measured using a complementary nucleotide sequence of the mRNA of the gene encoding Kindlin-1 and specifically hybridizing with the mRNA of the gene encoding Kindlin-1 or, a fragment thereof hybridizing specifically with the mRNA of the gene encoding Kindlin-1, this sequence or this fragment comprising 5 to 50 nucleotides, preferentially 10 to 20 nucleotides, or using a pair of primers or a probe of 10 to 60 nucleotides, preferentially 15 to 30 nucleotides comprising said sequence or said fragment. The level of expression may then be measured by any means known to those skilled in the art, for example by means of quantitative RT-PCR.

Within the scope of the invention, the terms “hybridize” or “hybridization”, are well-known to those skilled in the art, refer to the binding of a nucleic acid sequence with a particular nucleotide sequence under suitable conditions, particularly under stringent conditions.

The term “stringent conditions”, as used herein, corresponds to conditions suitable for producing bond pairs between the nucleic acids having a defined level of complementarity, while being unsuitable for the formation of pairs between the bonding nucleic acids having a lower complementarity than said defined level. The stringent conditions are dependent on hybridization and washing conditions. These conditions may be modified according to methods known to those skilled in the art. Generally, high-stringency conditions are a hybridization temperature approximately 5° C. less than the melting point (Tm), preferably close to the Tm of the perfectly base-paired strands. The hybridization procedures are well-known in the art.

High-stringency conditions generally involve hybridization at a temperature of approximately 50° C. to approximately 68° C. in a 5x SSC/5x Denhardt’s solution/1.0% SDS solution, and washing in a 0.2x SSC/0.1% SDS solution at a temperature between approximately 60° C. and approximately 68° C.

### Cancer Cells of the Patient

As used herein the term “patient” refers to a mammalian such as a rodent, a feline, an equine, a bovine, an ovine, a canine or a primate, and is preferably a primate and more preferably a human. The patient has been diagnosed as having a cancer and preferably a solid cancer, in particular an epithelial cancer. In an embodiment, the cancer is breast, lung, colon, pancreas, bladder, or head and neck cancer, preferably the cancer is a triple negative breast cancer. In an embodiment, the cancer is breast, pancreas, bladder, or head and neck cancer. In an embodiment, the cancer is pancreas, bladder, or head and neck cancer.

By “cancer cells”, it is meant cells extracted from a cancerous tumor of a patient. These cells can be alive or lysed. They can be processed (such as purification, fractionation, enzymatic processing, freezing etc...) prior to the measuring of the expression level of Kindlin-1.

### Reference Level

As used herein, the term “reference level” means an expression level of Kindlin-1 which is determined for the methods of the invention. In one particular embodiment, the reference level is determined by the mean value of the Kindlin-1 expression level in several cancer cells samples. In another embodiment, the reference level is the optimal threshold value determined by ROC analysis. Cancer cells samples used for the determination of the reference level are preferably of the same type of cancer as the cancer cells of said patient. Preferably, the cancer cells used for determining the reference level originate from the same organ and same cellular type (preferably epithelial cells, i.e. carcinoma) as the cancer cells of the patient used in the method according to the invention. According to the invention, a reference level may be determined by a plurality of samples, preferably more than 5, 50, 100, 200 or 500 samples.

In some embodiments, the reference level is determined from the expression level of Kindlin-1 measured in samples of cancer cells sensitive to EGFR/RAS pathway inhibitor therapy, wherein said cancer cells sensitive to EGFR/RAS pathway inhibitor therapy are of the same type as the cancer cells of said patient having cancer, and/or the reference level is determined from the expression level of Kindlin-1 measured in samples of cancer cells resistant to EGFR/RAS pathway inhibitor therapy, wherein said cancer cells resistant to EGFR/RAS pathway inhibitor therapy are of the same type as the cancer cells of said patient having cancer.

In particular, if the reference level is determined with the expression level of Kindlin-1 measured in samples of cancer cells sensitive to EGFR/RAS pathway inhibitor therapy, and the measured Kindlin-1 expression level in cancer cells of a patient having cancer is equal or above the reference level, then said patient can be considered eligible to EGFR/RAS pathway inhibitor therapy.

If the reference level is the determined with expression level of Kindlin-1 measured in samples of cancer cells resistant to EGFR/RAS pathway inhibitor therapy, and the measured Kindlin-1 expression level in cancer cells of said patient having cancer is above the reference level, then said patient can be considered eligible to EGFR/RAS pathway inhibitor therapy.

As used herein, the term “cancer cells sensitive to EGFR/RAS pathway inhibitor therapy” means cells extracted from cancerous tumors which have been successfully treated with an EGFR/RAS pathway inhibitor therapy and/or cancer cell lines which are known to be sensitive to EGFR/RAS pathway inhibitor therapy as determined by the IC₅₀ values (for example as can be obtained from Genomics of Drug Sensitivity in Cancer database). In particular, “cancer cells sensitive to EGFR/RAS pathway inhibitor therapy” means cells extracted from cancerous tumors that showed complete response (CR), partial response (PR), or were stable (stable disease SD) after EGFR/RAS pathway inhibitor therapy.

As used herein, the term “cancer cells resistant to EGFR/RAS pathway inhibitor therapy” means cells extracted from cancerous tumors which treatment with an EGFR/RAS pathway inhibitor therapy as no effect or no satisfactory effect. In particular, “cancer cells resistant to EGFR/RAS pathway inhibitor therapy” means cells extracted from cancerous tumors that showed progressive disease (PD) after EGFR/RAS pathway inhibitor therapy.

In a particular embodiment the reference level is determined by ROC analysis on samples of cancer cells sensitive to EGFR/RAS pathway inhibitor therapy extracted from cancerous tumors that showed complete response (CR), partial response (PR), or were stable (stable disease SD) after EGFR/RAS pathway inhibitor therapy compared to samples of cancer cells resistant to EGFR/RAS pathway inhibitor therapy which are extracted from cancerous tumors that showed progressive disease (PD) after EGFR/RAS pathway inhibitor therapy.

### Medical Indications

The invention also concerns an EGFR/RAS pathway inhibitor for use for treating cancer in a patient, wherein said patient has been identified as having a Kindlin-1 expression level in cancer cells equal to, or above, a reference level.

The invention also concerns a method of treatment of cancer in a patient comprising the administration of an EGFR/RAS pathway inhibitor, wherein said patient has been identified has having a Kindlin-1 expression level in cancer cells equal to, or above, a reference level.

The invention concerns an EGFR/RAS pathway inhibitor for use for treating cancer in a patient, wherein said patient is considered eligible to EGFR/RAS pathway inhibitor therapy, preferably according to the method for selecting a patient having cancer as eligible to EGFR/RAS pathway inhibitor therapy of the invention.

The invention concerns a method of treatment of cancer in a patient comprising the administration of an EGFR/RAS pathway inhibitor, wherein said patient is considered eligible to EGFR/RAS pathway inhibitor therapy, preferably according to the method for selecting a patient having cancer as eligible to EGFR/RAS pathway inhibitor therapy of the invention.

As used herein, the term “reference level” means an expression level of Kindlin-1 which is determined for the methods of the invention. In one particular embodiment, the reference level is determined by the mean value of the Kindlin-1 expression level in several cancer cells samples. In another embodiment, the reference level is the optimal threshold value determined by ROC analysis. Cancer cells samples used for the determination of the reference level are preferably of the same type of cancer as the cancer cells of said patient. Preferably, the cancer cells used for determining the reference level originate from the same organ and same cellular type (preferably epithelial cells, i.e. carcinoma) as the cancer cells of the patient used in the method according to the invention. According to the invention, a reference level may be determined by a plurality of samples, preferably more than 5, 50, 100, 200 or 500 samples.

In some embodiments, the reference level is determined from the expression level of Kindlin-1 measured in samples of cancer cells sensitive to EGFR/RAS pathway inhibitor therapy, wherein said cancer cells sensitive to EGFR/RAS pathway inhibitor therapy are of the same type as the cancer cells of said patient having cancer, and/or the reference level is determined from the expression level of Kindlin-1 measured in samples of cancer cells resistant to EGFR/RAS pathway inhibitor therapy, wherein said cancer cells resistant to EGFR/RAS pathway inhibitor therapy are of the same type as the cancer cells of said patient having cancer.

In particular, if the reference level is determined from the expression level of Kindlin-1 measured in samples of cancer cells sensitive to EGFR/RAS pathway inhibitor therapy, , and the measured Kindlin-1 expression level in cancer cells of said patient having cancer is equal or above the reference level, then said patient is treated by the administration of an EGFR/RAS pathway inhibitor.

In particular, if the reference level is determined from the expression level of Kindlin-1 measured in samples of cancer cells resistant to EGFR/RAS pathway inhibitor therapy, and the measured Kindlin-1 expression level in cancer cells of said patient having cancer is above the reference level, then said patient is treated by the administration of an EGFR/RAS pathway inhibitor.

In a particular embodiment the reference level is determined by ROC analysis on samples of cancer cells sensitive to EGFR/RAS pathway inhibitor therapy extracted from cancerous tumors that showed complete response (CR), partial response (PR), or were stable (stable disease SD) after EGFR/RAS pathway inhibitor therapy compared to samples of cancer cells resistant to EGFR/RAS pathway inhibitor therapy which are extracted from cancerous tumors that showed progressive disease (PD) after EGFR/RAS pathway inhibitor therapy.

### Method of Prognosis

The invention also concerns a method of prognosis cancer outcome or progression, in a patient suffering from EGFR/RAS-driven cancer, comprising:
a) Measuring Kindlin-1 expression level in cancer cells of said patient suffering from EGFR/RAS-driven cancer;
b) Determining that the cancer is likely to progress or have a poor outcome if the measured Kindlin-1 expression level in cancer cells of the patient is equal to, or above, a first reference level; or determining that the cancer is unlikely to progress or have a poor outcome if the measured Kindlin-1 expression level in cancer cells of the patient is equal to or below a second reference level.

By “prognosis cancer outcome or progression” it is meant evaluating the chances of success of the treatment or the chances to reduce, alleviate or inhibit or eliminate the cancer or symptoms of said cancer, evaluating the chances of degradation of the state of the patient, and in particular the chances of growth of the tumor and/or the apparition of metastasis. In preferred embodiment, it means evaluating the chances of survival and more preferably of metastasis-free survival.

The term “EGFR/RAS-driven cancer”, as used herein, corresponds to cancer wherein EGFR and/or RAS is highly expressed and/or cancer wherein EGFR and/or RAS are activated.

In some embodiment the method of prognosis also includes a step c), administering an anticancerous treatment if the patient suffering from EGFR/RAS-driven cancer has been determined as having a cancer likely to progress or with a poor outcome.

By “anticancerous treatment” it is meant any treatment used to treat cancer and in particular EGFR/RAS-driven cancer such as surgery, chemotherapy, immunotherapy or radiotherapy. In a particular embodiment, the anticancerous treatment is EGFR/RAS pathway inhibitor therapy and/or a Kindlin-1 inhibitor therapy.

### Kindlin-1 Inhibitor Therapy

By “Kindlin-1 inhibitor therapy”, it is meant a therapy using the administration of at least one Kindlin-1 inhibitor. Such inhibitor can be a direct inhibitor of Kindlin-1 activity or expression, i.e. which inhibits or reduces Kindlin-1 biological activity and/or reduces the amount of the Kindlin-1 protein. Therefore, the Kindlin-1 inhibitor may reduce or inhibit Kindlin-1 expression, or reduce or inhibit Kindlin-1 interaction ability with its targets.

“Kindlin-1 expression” also refers to events modifying Kindlin-1 mRNA transcriptionally or post-transcriptionally, by cleavage and maturation, to provide a functional Kindlin-1 ; it also includes events modifying Kindlin-1 protein during translation, as well as post-translational modifications.

An “inhibitor of Kindlin-1 expression” refers to any compound that has a biological effect to inhibit the expression of a Kindlin-1 gene and/or the expression of Kindlin-1 protein. In one embodiment of the invention, said inhibitor of Kindlin-1 expression is a short hairpin RNA (shRNA), a small inhibitory RNA (siRNA), or an antisense oligonucleotide. Preferably, the inhibitor of Kindlin-1 expression is a siRNA or a shRNA.

Short hairpin RNA (shRNA) or Small inhibitory RNAs (siRNAs) can function as inhibitors of gene expression for use in the invention. Gene expression can be reduced with a small double stranded RNA (dsRNA), or a vector or construct causing the production of a small double stranded RNA, such that gene expression is specifically inhibited (i.e. RNA interference or RNAi). Methods for selecting an appropriate dsRNA or dsRNA-encoding vector are well known in the art for genes whose sequence is known.

Inhibitors of Kindlin-1 for use in the invention may be based on antisense oligonucleotide (ODNs) constructs. Antisense oligonucleotides, including antisense RNA molecules and antisense DNA molecules, would act to directly block the activity of Kindlin-1 by binding to Kindlin-1 mRNA and thus preventing binding, leading to mRNA degradation. Methods for using antisense techniques for specifically inhibiting gene expression of genes whose sequence is known are well known in the art. Antisense oligonucleotides useful as inhibitors of Kindlin-1 can be prepared by known methods. These include techniques for chemical synthesis such as, e.g., by solid phase phosphoramadite chemical synthesis. They can also be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters.

As used herein, the terms “inhibitor of the interaction” means preventing or reducing the direct or indirect association of one or more molecules, nucleic acids, peptide or proteins.

As used herein, the term “inhibitor of Kindlin-1” encompasses molecules that can prevent the interaction of Kindlin-1 with its target, by competition or by fixing to one of the molecules. Preferably, the inhibitor is a chemical molecule, peptide, protein, aptamer, antibody or antibody fragment. In a particular embodiment, the inhibitor is an antibody or an antibody fragment.

By “peptide”, it is meant an amino acid sequence comprising from 2 to 30 amino acids. By “protein”, it is meant an amino acid sequence comprising at least 31 amino acids, preferably 50 to 500 amino acids.

By “antibody” it is meant immunoglobulin molecules and immunologically active portions of immunoglobulin molecules, i.e., molecules that contain an antigen binding site which immunospecifically binds an antigen. As such, the term antibody encompasses not only whole antibody molecules, but also antibody fragments as well as variants (including derivatives) of antibodies and antibody fragments. In particular, the antibody according to the invention may correspond to a polyclonal antibody, a monoclonal antibody (e.g. a chimeric, humanized or human antibody), a fragment of a polyclonal or monoclonal antibody or a diabody.

By “antibody fragments” it is meant a portion of an intact antibody, preferably the antigen binding or variable region of the intact antibody. Examples of antibody fragments include Fv, Fab, F(ab′)₂, Fab′, Fd, dAb, dsFv, scFv, sc(Fv)₂, CDRs, diabodies and multi-specific antibodies formed from antibodies fragments.

By “aptamers” it is meant the class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library. The random sequence library is obtainable by combinatorial chemical synthesis of DNA. Possible modifications, uses and advantages of this class of molecules have been reviewed in Jayasena S.D., Clin. Chem., 1999, 45(9):1628-50. Then, after identifying the aptamers directed against Kindlin-1 as described above, those skilled in the art can readily select the ones inhibiting Kindlin-1. Preferably, the aptamer is an oligonucleotide or polypeptide from 10 to 30 kDa.

Methods for determining whether a compound is a Kindlin-1 inhibitor are well-known by the person skilled in the art. For example, the person skilled in the art can assess whether a compound decreases Kindlin-1 expression.

Other methods for determining whether a compound is an inhibitor of Kindlin-1 may be for example, by measuring the biological activity of Kindlin-1 , through measuring one of the phenomenon in which Kindlin-1 is known to play a role.

As used herein, the term “reference level” means an expression level of Kindlin-1 which is determined for the methods of the invention. In one particular embodiment, the reference level is determined by the mean value of the Kindlin-1 expression level in several cancer cells samples. In another embodiment, the reference level is the optimal threshold value determined by ROC analysis. Cancer cells samples used for the determination of the reference level are preferably of the same type of cancer as the cancer cells of said patient. Preferably, the cancer cells used for determining the reference level originate from the same organ and same cellular type (preferably epithelial cells, i.e. carcinoma) as the cancer cells of the patient used in the method according to the invention. According to the invention, a reference level may be determined by a plurality of samples, preferably more than 5, 50, 100, 200 or 500 samples.

In some embodiments, the first reference level is determined from the expression level of Kindlin-1 measured in samples of cancer cells of aggressive EGFR/RAS driven cancers, wherein said cancer cells of aggressive EGFR/RAS driven cancers are of the same type as the cancer cells of said patient having cancer, and/or the second reference level is determined from the expression level of Kindlin-1 measured in samples of cancer cells of non-aggressive EGFR/RAS driven cancers, wherein said cancer cells of non-aggressive EGFR/RAS driven cancers are of the same type as the cancer cells of said patient having cancer.

In particular, in the method of prognosis cancer outcome or progression according to the invention, the first reference level can be determined from the expression level of Kindlin-1 measured in samples of cancer cells of aggressive EGFR/RAS-driven cancers, if the measured Kindlin-1 expression level in cancer cells of said patient having cancer is equal or above this reference level, then said patient cancer is likely to progress or have a poor outcome.

In particular, in the method of prognosis cancer outcome or progression according to the invention, the second reference level can be determined from the expression level of Kindlin-1 measured in samples of cancer cells of non-aggressive EGFR/RAS-driven cancer, if the measured Kindlin-1 expression level in cancer cells of said patient having cancer is below this second reference level, then said patient cancer is unlikely to progress or have a poor outcome.

In some embodiments, the first and the second reference level are the same.

As used herein, the term “cancer cells of aggressive EGFR/RAS-driven cancer” means cells extracted from cancerous tumors which have rapidly progressed in a patient. In a particular embodiment, “cancer cells of aggressive EGFR/RAS-driven cancer” means cells extracted from cancerous tumors that rapidly produce metastasis, i.e. that produce metastasis in less than one year and preferably less than 6 months.

As used herein, the term “cancer cells of non-aggressive EGFR/RAS driven cancer” means cells extracted from cancerous tumors which stay stable or which size reduced without forming new metastasis or which was successfully treated.

According to an embodiment in the method of prognosis cancer outcome or progression, the patient is suffering from a bladder or a head and neck cancer.

The invention also concerns an EGFR/RAS pathway inhibitor and/or a Kindlin-1 inhibitor for use for treating cancer in a patient suffering from EGFR/RAS-driven cancer, wherein said patient has been identified as having a cancer likely to progress or having a poor outcome, preferably said patient has been identified according to the method of prognosis cancer outcome or progression of the invention.

In an embodiment, the EGFR/RAS-driven cancer is a solid cancer, in particular an epithelial cancer. In an embodiment, the EGFR/RAS-driven cancer is a breast, lung, colon, pancreas, bladder, or head and neck cancer, preferably a triple negative breast cancer. In an embodiment, the EGFR/RAS-driven cancer is a breast, pancreas, bladder, or head and neck cancer. In an embodiment, the EGFR/RAS-driven cancer is pancreas, bladder, or head and neck cancer.

The invention will be further illustrated by the following figures and examples.

### FIGURES

FIG. 1A depicts a scatter plot representing the differential mRNA expression levels of Kindlin-1 in EGFR/RAS driven breast cancer cells versus non EGFR/RAS driven cells. Mean ±SE represented. Statistical analysis were performed using the Mann-Whitney test (****p<0,0001 ).

FIG. 1B depicts western blot results performed in a subset of breast cancer cell lines to compare Kindlin-1 expression with the activation of EGFR/RAS pathway at a protein level.

FIG. 2 depicts western blot performed in a subset of lung and pancreatic cancer cell lines to compare Kindlin-1 expression with the activation of EGFR/RAS pathway at a protein level.

FIG. 3 depicts a scatter plot showing Kindlin-1 protein expression in low versus high EGFR expression. Each point represents the H-score from a single tissue sample ranging from total absence of Kindlin-1 in the epithelial compartment (H-score 0), to very strong Kindlin-1 staining (H-score 300). Mean ±SE represented.

FIG. 4 depicts Box and whisker plots representing the differential mRNA expression levels of Kindlin-1 in EGFR/RAS driven tumors versus non EGFR/RAS driven tumors in pancreas, lung, bladder, head and neck and colon human tumors. Mean ±SE represented. Statistical analysis were performed using the Mann-Whitney test (****p<0.0001 ; ***p<0.001; **p<0.01 ).

FIG. 5 depicts Kaplan-Meier plots showing metastasis free survival of triple negative breast (TNBC), lung, pancreatic, bladder, head and neck and colon cancer patients for the expression of Kindlin-1 taking into account the activation of EGFR pathway and/or KRAS, HRAS, BRAF, EGFR mutational status. Statistical analysis were performed by a Log-rank test.

FIG. 6 depicts a scatter plot representing the differential mRNA expression levels of Kindlin-1 in low sensitive breast cancer cells versus high sensitive cells to different EGFR/RAS pathway inhibitors compared to a standard drug used in breast cancer treatment (palbociclib). Mean ±SE represented. Statistical analysis were performed using the Mann-Whitney test (**p<0.01; *p<0.05; ns: not significant).

FIG. 7 depicts a scatter plot representing the differential mRNA expression levels of Kindlin-1 in low sensitive cancer cells versus high sensitive cells to different EGFR/RAS pathway inhibitor, respectively in lung cancer, pancreas cancer, bladder cancer, head and neck cancer and colon cancer. Mean ±SE represented. Statistical analysis were performed using the Mann-Whitney test.

FIG. 8A depicts western blot of Kindlin-1 protein in 27 triple-negative breast cancer PDX models.

FIG. 8B depicts Pearson correlation was calculated between tumor growth inhibition (TGI) and Kindlin-1 protein expression in 15 PDX models (R= 0.58, p<0.02).

FIG. 8C depicts graphs showing relative tumor growth after Selumetinib treatment of two PDX models highly expressing Kindlin-1 (#965 and #138) and 2 PDX models with a low Kindlin-1 expression (#73 and #408).

FIG. 9A depicts box and whisker plot representing the differential mRNA expression levels of Kindlin-1 in head and neck cancer patients non responding versus head and neck cancer patients responding to Cetuximab monotherapy (p=0.01).

FIG. 9B depicts box and whisker plot representing the differential disease free survival between patients expressing low versus high levels of Kindlin-1 (p=0.2) in head and neck cancer patients. Mean ±SE represented. Statistical analysis were performed using the Mann-Whitney test.

FIG. 10 depicts graphs representing cell viability at 48h and 72 h as a function of the drug concentration M, for MDA MB 468 control cells (w) or stably overexpressing Kindlin-1 (k1).

FIG. 11 depicts box and whisker plot representing the differential mRNA expression levels of Kindlin-1 in patients with a long progression free survival (PFS) versus patients with a short progression free survival (p=0.004) on a cohort of EGFRwt/KRASwt NSCLC patients treated with erlotinib (Byers et al., 2012 cohort). Statistical analysis were performed using the Mann-Whitney test.

FIG. 12A depicts box and whisker plot representing the differential mRNA expression levels of Kindlin-1 in head and neck cancer patients with short progression free survival versus long progression free survival following Cetuximab treatment (p=0.001), on a cohort of HNSCC patients treated with cetuximab (Bossi et al., 2016 cohort). Mean ±SE represented. Statistical analysis were performed using the Mann-Whitney test.

FIG. 12B depicts box and whisker plot representing the differential mRNA expression levels of Kindlin-1 in metastatic CRC patients with a progressive disease versus patients with disease control treated with Cetuximab (p=0.044) on a cohort of Metastatic CRC patients treated with cetuximab (Khambata-Ford et al., 2007 cohort). Mean ±SE represented. Statistical analysis were performed using the Mann-Whitney test.

### EXAMPLES

### Example 1: Materials and Methods

### Gene Set Enrichment Analysis (GSEA)

Microarray expression of cancer cell lines from the Cancer Cell Line Encyclopedia (CCLE) dataset publicly available from cBioPortal (www.cbioportal.org/) were analyzed by GSEA v4.0.2 software (58 breast, 174 lung, 40 pancreas, 24 bladder, 30 head and neck, 44 colon cancer cell lines). Gene sets used for the analysis were obtained from the Molecular Signatures Database (MSigDB 7.0; http://software.broadinstitute.org/gsea/msigdb/). We compared expression profiles of cell lines with a high Kindlin-1 expression versus cell lines with a low Kindlin-1 expression. The optimal cutoff point to categorize cell lines into high versus low Kindlin-1 expression groups was determined performing a hierarchical clustering on Kindlin-1 expression level. GSEA plots showed specific gene sets significantly enriched in cancer cell lines with a high mRNA expression of Kindlin-1.

### Cell Culture

Human breast cancer cell lines DU4475, MDA-MB-231, MDA-MB-468, HCC-1954, HCC-1569, MDA-MB-415, MDA-MB-361, BT549, MDA-MB-436, MCF7 and HS578T; human lung cancer cell lines H727, H358, H1975, A549, H69, H1650 and CALU1 ; and human pancreatic cancer cell lines HPAFII, ASPC1, MIAPACA2 and PANC1 were purchased from ATCC (Manassas,VA,USA), maintained at 37° C. with or without 5% CO₂ and grown in DMEM, RPMI 1640, MEM Alpha, McCoy’s or Leibovitz’s L-15 medium supplemented with 10% FBS and 1% antibiotics (50 µg/mL penicillin, 50 µg/mL streptomycin, 100 µg/mL neomycin).

### Western Blotting

Cells were lysed using RIPA buffer (50 mM Tris-HCl, pH 8; 150 mM NaCl; 0.5% triton; 0.5% deoxycholic acid) containing protease inhibitors (1:1000 orthovanadate, 1:1000 apoprotinine, 1:200 PMSF). Protein extracts were loaded on a polyacrylamide gel, transferred to a nitrocellulose membrane and incubated overnight at 4° C. with primary antibodies for Kindlin-1 (1:1000), pMEK½ (1:1000, 9154P, Cell Signaling, Danvers, MA) or MEK½ (1:1000, 9126S, Cell Signaling). GAPDH (1:500, Clone V18, Santa Cruz Biotechnology) or b-actin (1:16000, Clone AC15, Sigma-Aldrich) were used as loading controls. The signals were detected according to the ECL Western Blotting Analysis System procedure (GE Healthcare, Buckinghamshire, UK).

### Human Breast, Lung, Pancreas, Urinary Tract, Head and Neck and Colon Tumors

Publicly available data set from the TCGA were analyzed for different human cancer types (n= 1084 breast tumors, 566 lung tumors, 184 pancreatic tumors, 411 urinary tract tumors, 488 head and neck tumors and 524 colon tumors obtained from cBioPortal (www.cbioportal.org/). Kindlin-1 and EGFR mRNA expression and/or amplification and the mutational status of KRAS, NRAS, HRAS, EGFR and BRAF were examined for each set of cancers.

Tumor sections of 62 breast tumors (tissue microarray) and 96 lung adenocarcinomas and adjacent normal breast tissues from patients treated at the Institut Curie were obtained from the Pathology Department of Hospital Curie. For the semi-quantitative analysis of Kindlin-1 and EGFR protein expression, the H-scored method assigned a score of 0-300 to each patient, based on the percentage of cells stained at different intensities. For lung tumors, EGFR and KRAS mutational status had been determined.

A series of 18 head and neck patients undergoing Cetuximab monotherapy at the Hôpital Curie was also analyzed at the RNA level.

### Breast Cancer Patient Derived Xenografts (PDX)

27 triple negative breast cancer PDX models were obtained from the Laboratoire d′Investigation Pre-clinique (Institut Curie) as previously described. Informed consent was obtained from patients before xenograft establishment. When tumors reached a volume of 60 to 200 mm³, mice were randomly assigned to the control or treated groups. Each group of treatment consisted of 3 or 4 mice. Selumetinib was purchased from MedChemExpress. It was administrated orally at a dose of 50 mg/kg 5x/week. Experiments complied with the current laws of France and were approved by Institut Curie ethical committee.

Tumor growth inhibition (TGI) was calculated using the following formula [1-(V_{ft}-V₀ₜ)/(V_{fc}-V_{0c}))]*100 where V_{ft} = final volume of the treated group (at the end of the treatment); V₀ₜ = initial volume of the treatment group (at the beginning of the treatment); V_{fc}= final volume of the control group (at the end of the treatment); V_{0c} = initial volume of the control group (at the beginning of the treatment).

### Statistical Analysis

Statistical analyses were performed with Prism, version 5 and 8.3.1 (GraphPad Software Inc.). Survival distributions were estimated by the Kaplan Meier method using PASW Statistics (version 18.0; SPSS Inc.)

### Results

### Kindlin-1 Expression Is Associated With the Activation of EGFR/RAS Pathway In Breast Cancer Cell Lines

To identify signaling pathways associated with Kindlin-1 overexpression in human breast cancer, we screened the microarray expression of 58 breast cancer cell lines from the Cancer Cell Line Encyclopedia (CCLE) dataset. A gene set enrichment analysis (GSEA) was used to detect the pathways differentially enriched in cell lines with a high Kindlin-1 expression (19 cell lines) versus cell lines with a low Kindlin-1 expression (39 cell lines). GSEA plots demonstrated that EGFR/RAS pathway is enriched in breast cancer cell lines with a high mRNA expression of Kindlin-1. We highlighted several enriched gene sets from the EGFR/RAS pathway: MEK upregulation, EGFR upregulation, KRAS signaling and KRAS dependency.

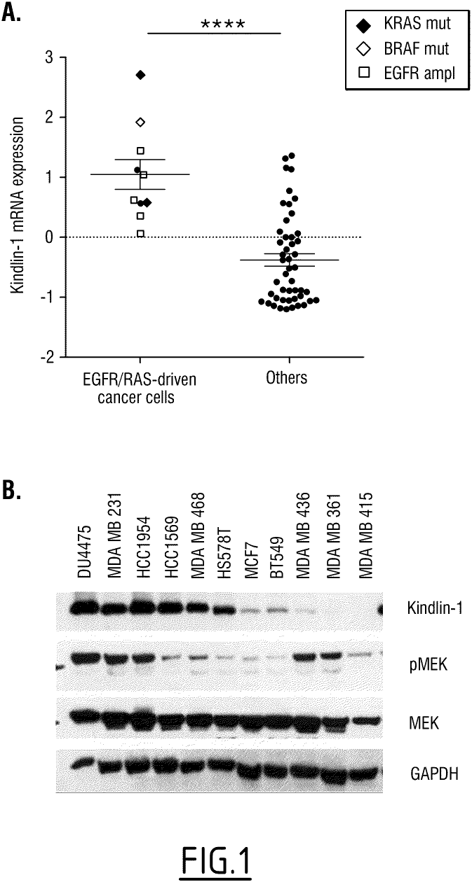

Next, we analyzed the correlation between Kindlin-1 expression and the EGFR/RAS altered status in these 58 breast cancer cell lines from CCLE. RNA expression levels of Kindlin-1 were examined in EGFR/RAS-driven breast cancer cells versus non EGFR/RAS-driven cells. Scatter plots (FIG. 1A) showed that high Kindlin-1 mRNA expression is associated to the activation of the EGFR/RAS pathway in breast cancer (p<10⁻⁴).

To test whether high kindlin-1 expression is associated with the activation of EGFR/RAS pathway at the protein level, we evaluated phosphorylation levels of MEK, one of the key downstream effectors of the EGFR/RAS pathway in a panel of breast cancer cell lines (n=11). We found that Kindlin-1 protein levels highly correlated with the phosphorylation levels of MEK (FIG. 1B).

### Kindlin-1 Expression Is Associated With EGFR/RAS Pathway Activation in Lung, Pancreas, Bladder, Head and Neck and Colon Cancers

Since Kindlin-1 overexpression has also been described in other solid tumors and its up regulation suggested decreasing patient survival in different cancer types. We further wanted to evaluate whether Kindlin-1 could be associated to the EGFR/RAS pathway not only in breast but also in a broader range of epithelial cancers. First, as we did with breast cancer cell lines, the microarray expression of 174 lung, 40 pancreatic, 24 bladder, 30 head and neck and 44 colon cancer cell lines from the Cancer Cell Line Encyclopedia (CCLE) dataset were examined by a gene set enrichment analysis (GSEA) to detect the signaling pathways differentially enriched in cell lines with a high Kindlin-1 expression versus cell lines with a low Kindlin-1 expression. GSEA plots demonstrated that EGFR/RAS pathway is also enriched in broad range of cancer cell lines with a high mRNA expression of Kindlin-1 , including lung, pancreas, bladder, head and neck and colon cancer cell lines.

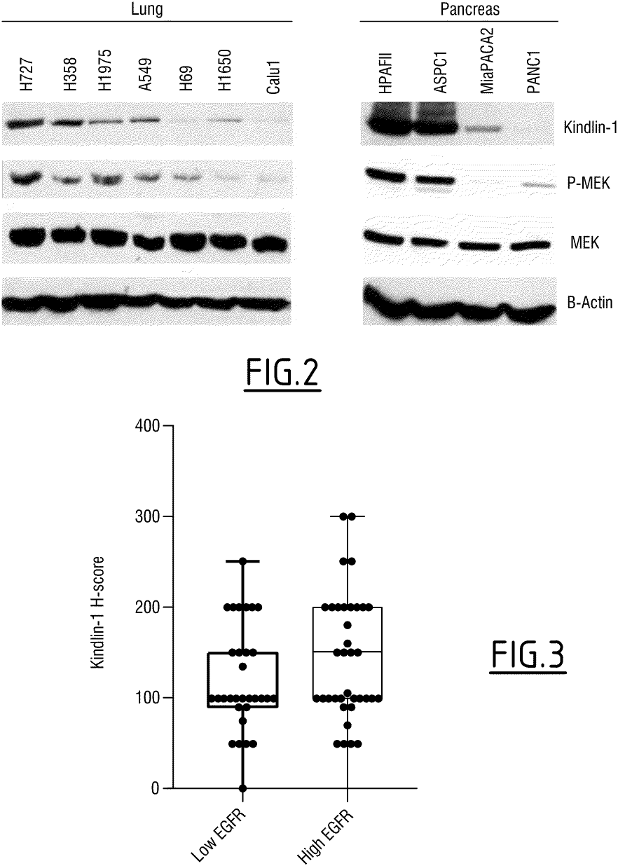

We then examined protein expression in several lung and pancreatic cancer cell lines. As we observed in breast cancer cell lines, we found that Kindlin-1 protein levels highly correlated with phosphorylation levels of MEK, indicating an activation of EGFR/RAS pathway specifically in these cell lines expressing high levels of Kindlin-1 (FIG. 2).

### Kindlin-1 Expression Is Associated With the Activation of EGFR/RAS Pathway In Human Breast Cancers

To further determine whether Kindlin-1 expression is associated with the EGFR/RAS pathway in human tumors, we analyzed Kindlin-1 and EGFR transcripts in a cohort of breast cancer patients for whom expression and mutational profiles are publicly available from the TCGA project (n=1084). We found that Kindlin-1 mRNA expression is correlated with EGFR mRNA expression (R²=0.45, p=0.02) supporting our results in cell lines. In addition, as found in cell lines, Kindlin-1 mRNA expression was associated not only with EGFR upregulation but also with the EGFR amplification and activating mutations in major drivers of the EGFR/RAS signaling axis (KRAS, HRAS, NRAS and BRAF genes) (p<0.0001).

We next performed an immunohistochemical analysis to evaluate Kindlin-1 and EGFR expression at a protein level in human breast tumors. We analyzed a tissue microarray (TMA) consisting of 62 human breast tumors. We compared the expression of both proteins in the same series of patients. Once again, we found a higher Kindlin-1 expression in those tumors highly expressing EGFR (FIG. 3).

### Kindlin-1 Expression Is Associated With the Activation of EGFR/RAS Pathway In Human Lung, Pancreas, Bladder and Head and Neck Cancers

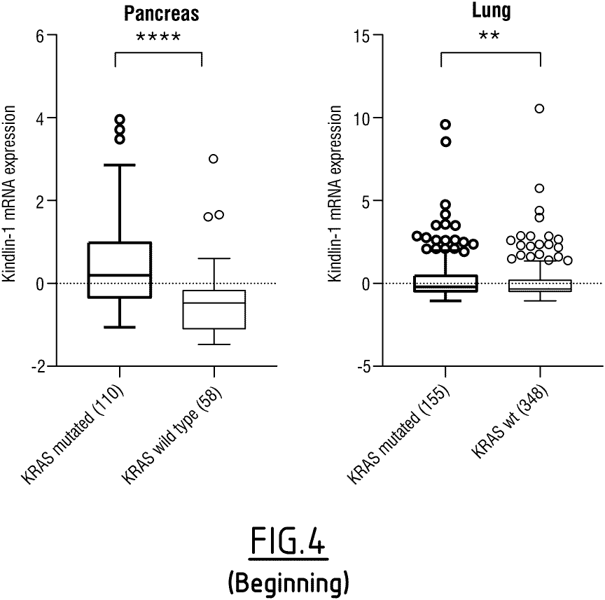

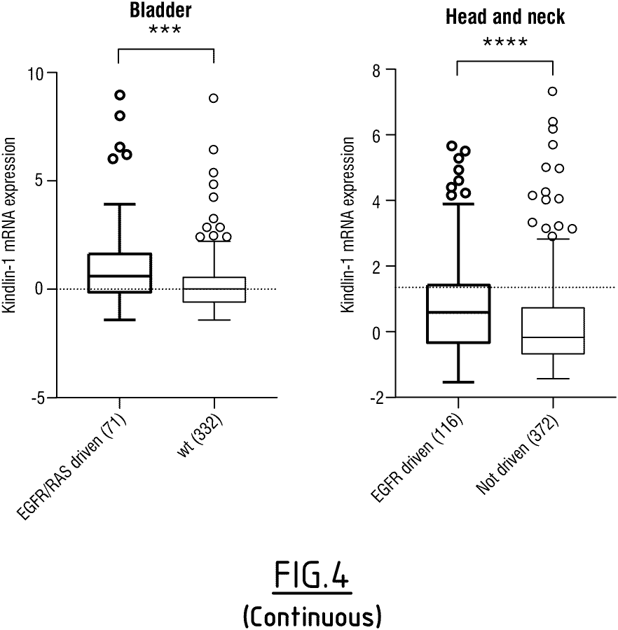

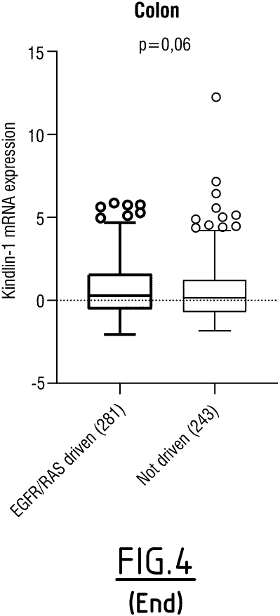

Next, we examined Kindlin-1 mRNA expression in a series of human lung (n=566), pancreatic (n=184), bladder (n=411), head and neck (n=488) and colon (n=524) tumors using publicly available data from the TCGA project. KRAS, HRAS, NRAS, BRAF activating mutations and EGFR mutations and/or amplification in those tumors were interrogated from the cbioportal database (FIG. 4). We found that Kindlin-1 expression was higher in KRAS-driven lung and pancreatic carcinomas versus the wild type ones. In the case of tumors from the bladder, those tumors with activating mutations of KRAS, HRAS, NRAS or BRAF and EGFR amplifications had increased levels of Kindlin-1 mRNA. Head and neck tumors with EGFR amplification and overexpression also showed higher Kindlin-1 expression levels. Finally, colon tumors with KRAS mutations and EGFR overexpression tended to have higher levels of Kindlin-1, even though the statistical significance was not reached (FIG. 4).

To corroborate these results, we investigated the expression of Kindlin-1 protein in a series of 96 lung adenocarcinomas with respect to their EGFR and KRAS mutation status. Immunohistochemical analyses showed a higher expression of Kindlin-1 in those tumors mutated for EGFR/RAS pathway.

Altogether, our findings suggest that Kindlin-1 expression is associated with EGFR/RAS pathway activation in a broad range of epithelial cancers.

### Kindlin-1 Expression Identifies EGFR/RAS-Driven Cancer Patients With Worst Survival Outcome

We then investigated the role of Kindlin-1 expression in patient survival using the TCGA data sets that are large enough to stratify patients based on EGFR and KRAS status. For breast cancers, Kindlin-1 expression was found to discriminate a subset of triple-negative breast cancer tumors, (the only subtype reported as EGFR-driven) with a worst clinical outcome (Log-rank test, p=0.003).

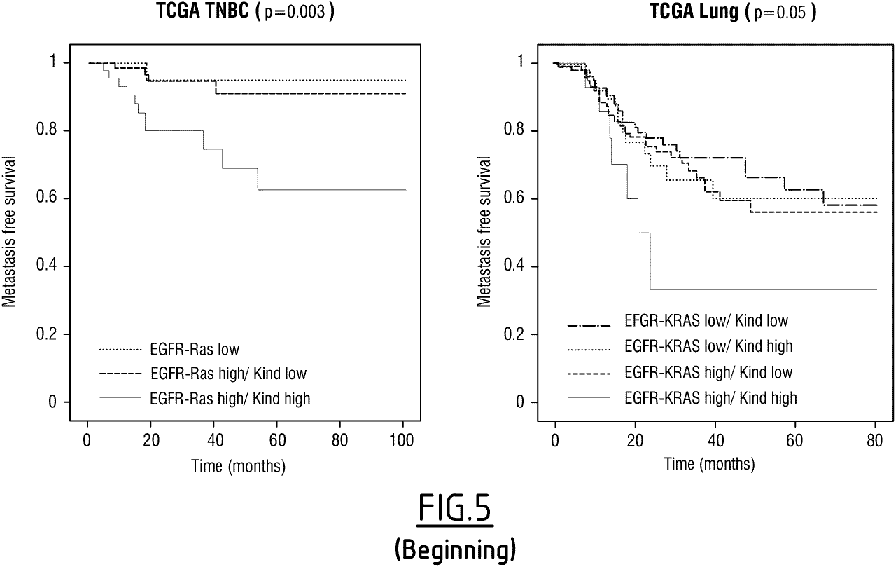

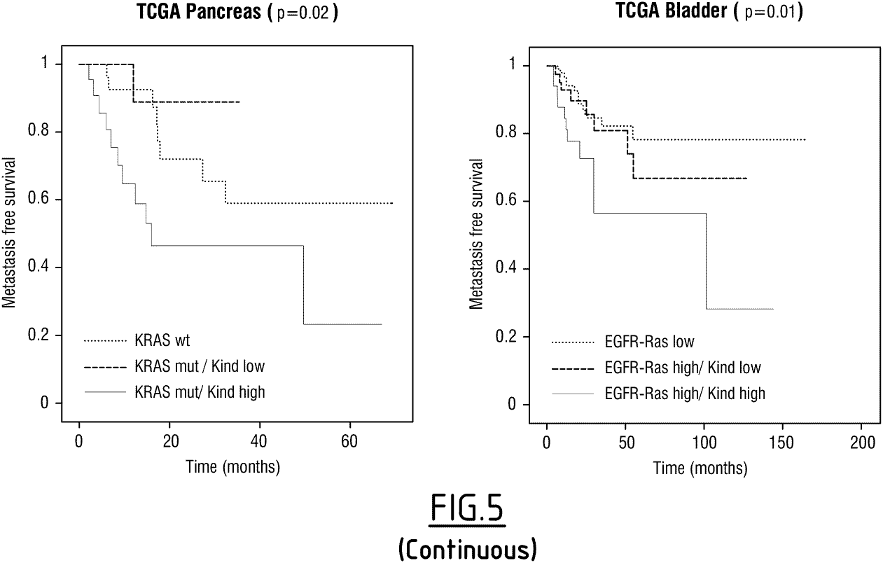

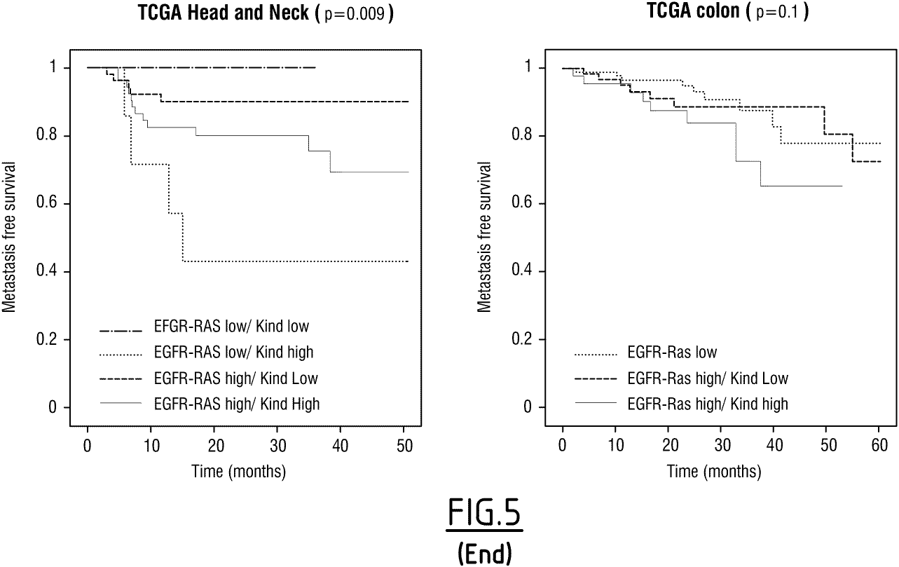

In the other cancer types, since EGFR overexpression/amplification and KRAS, EGFR, HRAS and BRAF mutations are usually mutually exclusive, we took into account all these drivers to define the EGFR-RAS axis high or low status. Kaplan-Meier curves are represented for each cancer type (FIG. 5). We found that high expression of Kindlin-1 is associated with a decreased metastasis-free survival in patients harboring EGFR-RAS driven tumors. Thus, Kindlin-1 expression identifies RAS/EGFR-driven cancer patients with the worst survival outcome.

### Kindlin-1 Expression Is Associated With Sensitivity to EGFR/RAS Pathway Inhibition in Breast Cancer Cells

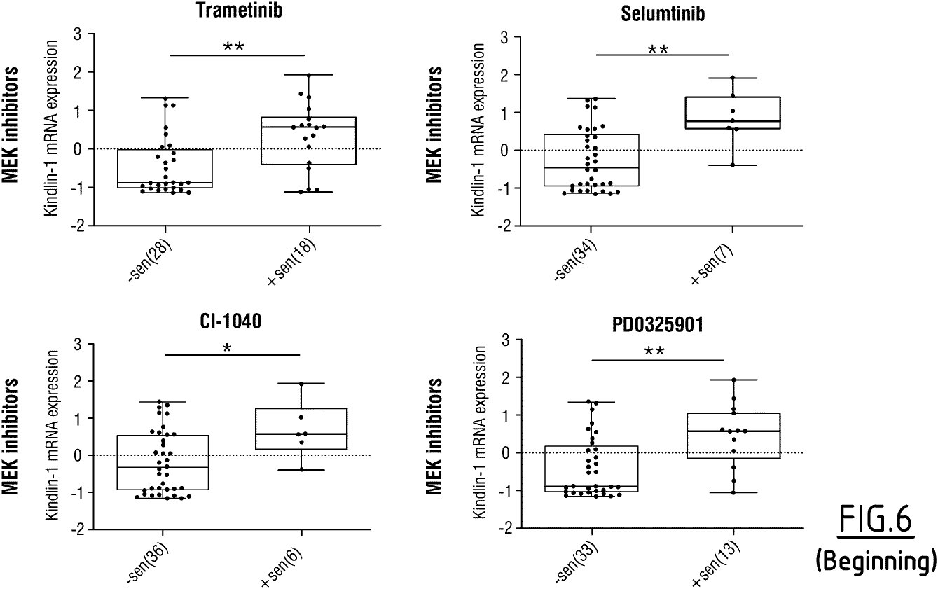

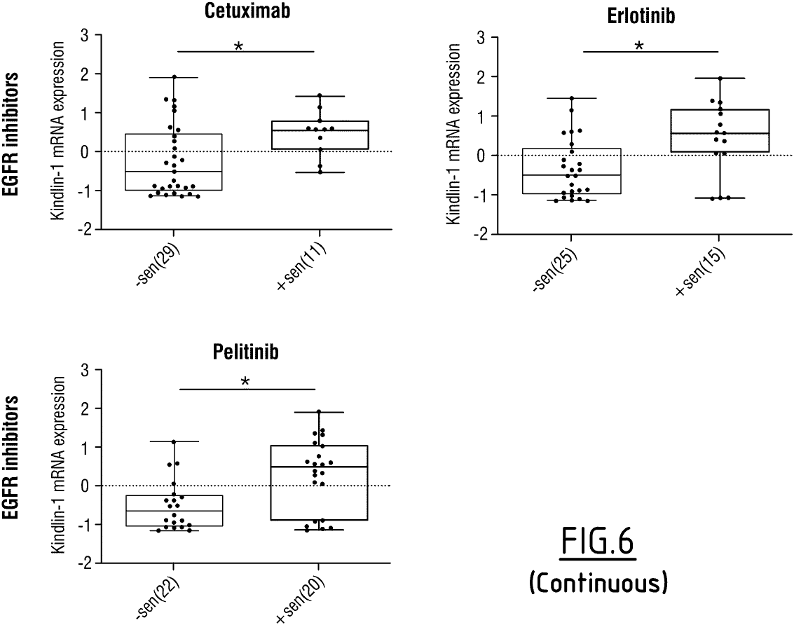

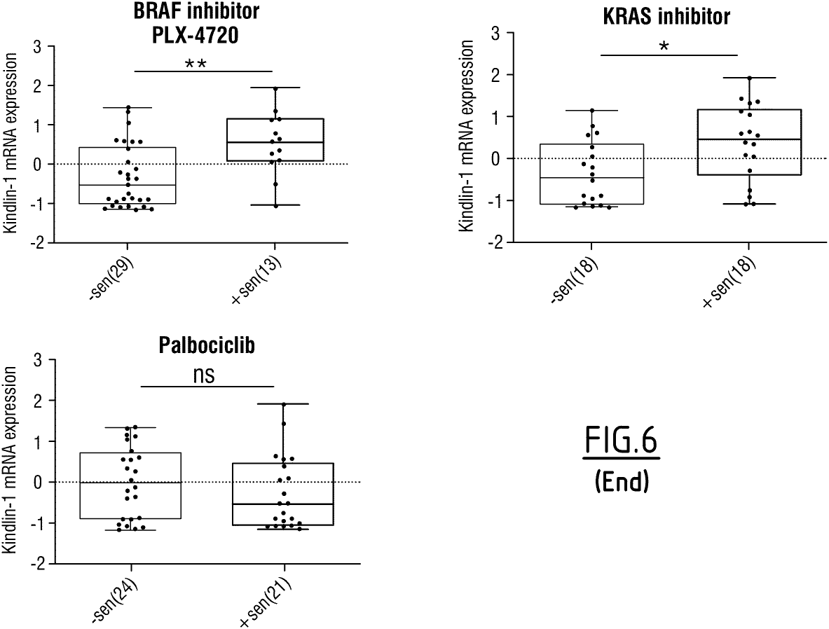

We next wanted to evaluate whether Kindlin-1 could be predictive of the response to inhibitors of the EGFR/RAS pathway in breast cancer. IC₅₀ values of different drugs were obtained from Genomics of Drug Sensitivity in Cancer database (www.cancerrxgene.org/) for the 58 breast cancer cell lines from CCLE. We demonstrated that Kindlin-1 mRNA expression was significantly increased in cell lines more sensitive to MEK inhibitors (Trametinib, p=0.0063; Selumetinib, p=0.0035; CI-1040, p=0.044 and PD0325901, p=0.0034), EGFR inhibitors (Cetuximab, p=0.017; Erlotinib, p=0.015 and Pelitinib, p=0.011), BRAF inhibitor (PLX-4720, p=0.005), and KRAS inhibitor (KRAS Inhibitor-12, p=0.016) compared with resistant cell lines. Moreover, this effect was specific to EGFR/RAS/MAPK inhibitors as there was no correlation between Kindlin-1 mRNA expression and Palbociclib, a CDK inhibitor used in breast cancer therapy (FIG. 6).

### Kindlin-1 Expression Is Associated With Sensitivity to EGFR/RAS Pathway Inhibitors in a Broad Range of Epithelial Cancers

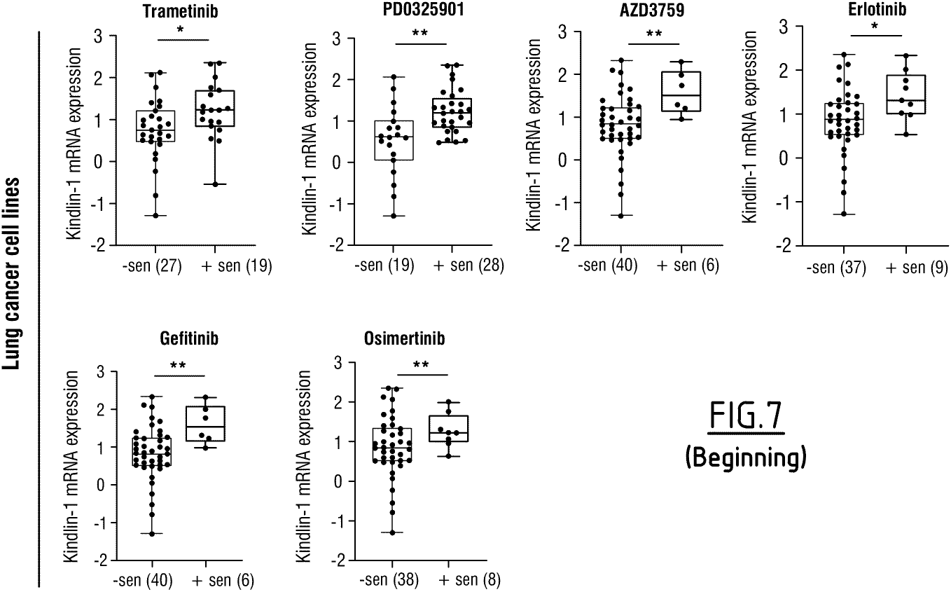

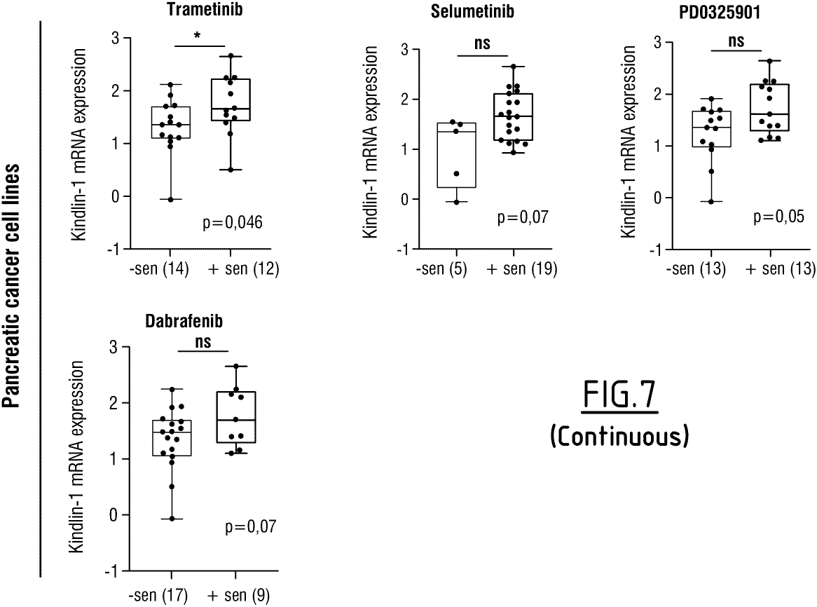

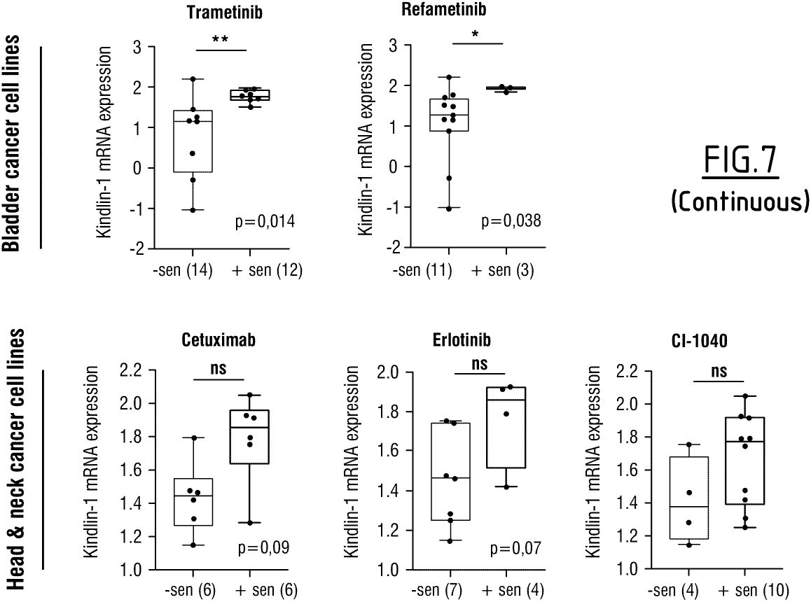

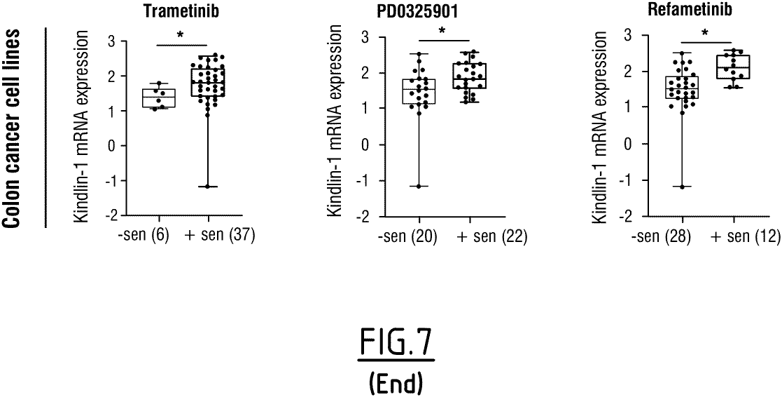

We next evaluated whether Kindlin-1 could be used as a predictive biomarker of the response to inhibitors of the EGFR/RAS pathway not only in breast but also in other epithelial cancers. Microarray expressions of 120 lung, 41 pancreatic, 21 bladder, 54 colon and 12 head and neck cancer cell lines from the CCLE dataset were analyzed using the IC₅₀ values obtained from Genomics of Drug Sensitivity in Cancer database as we did for breast cancer. We demonstrated that Kindlin-1 mRNA expression was significantly increased in lung cancer cell lines sensitive to MEK inhibitors (Trametinib, p=0.03; PD0325901, p=0.0018) and EGFR inhibitors (AZD3759, p=0.0098; Erlotinib, p=0.02; Gefitinib, p=0.0098 and Osimertinib, p=0.0032); pancreatic cancer cell lines more sensitive to MEK inhibitors (Trametinib, p=0.046; Selumetinib, p=0.07; PD0325901, p=0.05) and BRAF inhibitor Dabrafenib (p=0.07); urinary tract cancer cell lines more sensitive to MEK inhibitors (Trametinib, p=0.014 and Refametinib, p=0.038) and colon cancer cell lines more sensitive to MEK inhibitors (Trametinib, p=0.03; PD0325901, p=0.02 and Refametinib, p=0.001). Kindlin-1 mRNA expression had also a tendency to be increased in head and neck cancer cell lines more sensitive to EGFR inhibitors (Cetuximab, p=0.09 and Erlotinib p=0.07) and to the MEK inhibitor CI-1040 (p=0.1) (FIG. 7).

### Kindlin-1 Expression Is Associated With Sensitivity to Selumetinib in Breast Cancer in Vivo

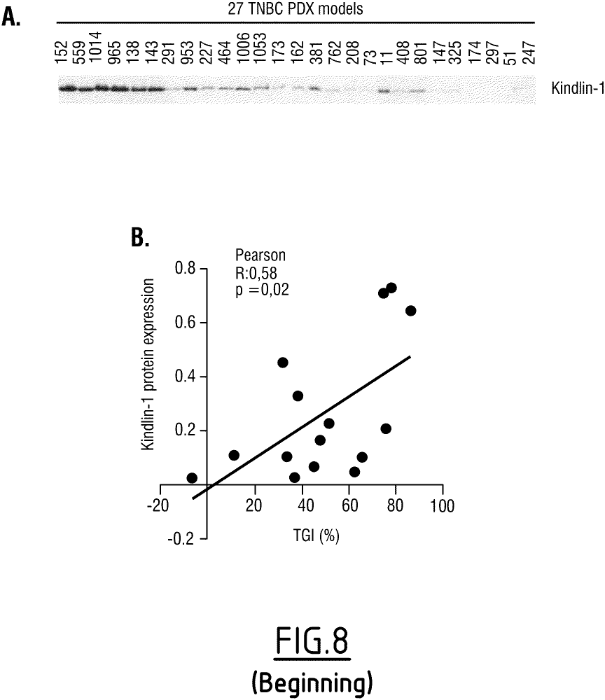

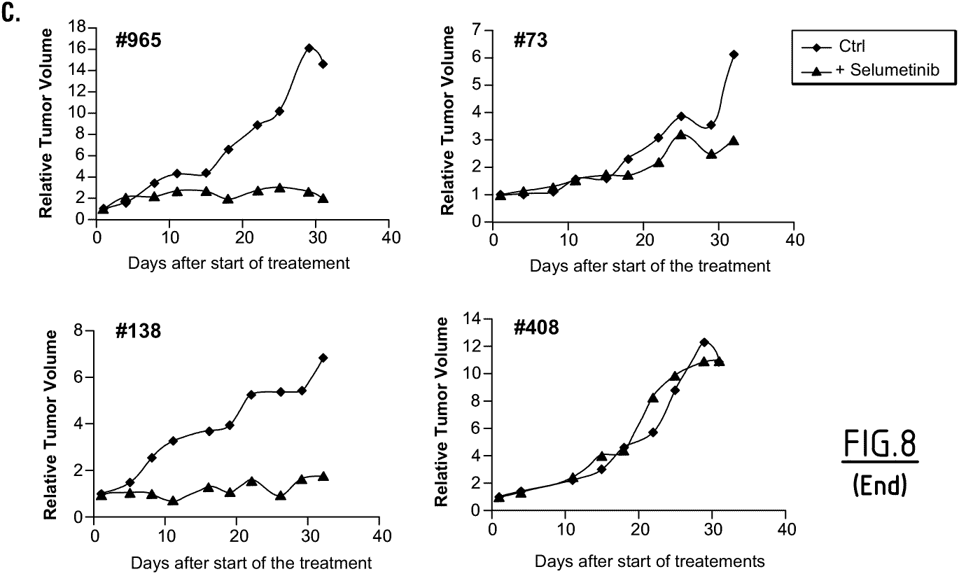

To further investigate whether Kindlin-1 expression could have a biomarker relevance, a cohort of 27 triple negative breast cancer patient derived xenografts (PDX) was used to test the correlation between Kindlin-1 expression and the response to MEK inhibitors. Kindlin-1 protein expression was assessed by western blots (FIG. 8A). The PDX were then treated with 50 mg/Kg of the Selumetinib, 5 days per week during 5 weeks. Tumor growth inhibition (TGI expressed in %) was evaluated with regard to the Kindlin-1 expression in each PDX model (FIG. 8B). We found a statistically significant correlation (Pearson r= 0.58; p=0.02) between Kindlin-1 protein expression and tumor growth inhibition (FIG. 8B). Selumetinib treatment had an important effect inhibiting tumor growth in PDXs highly expressing Kindlin-1 protein, not observed in PDXs with a lower Kindlin-1 expression (FIG. 8C).

### Kindlin-1 Expression Is Predictive of Cetuximab Response in Head and Neck Cancer Patients

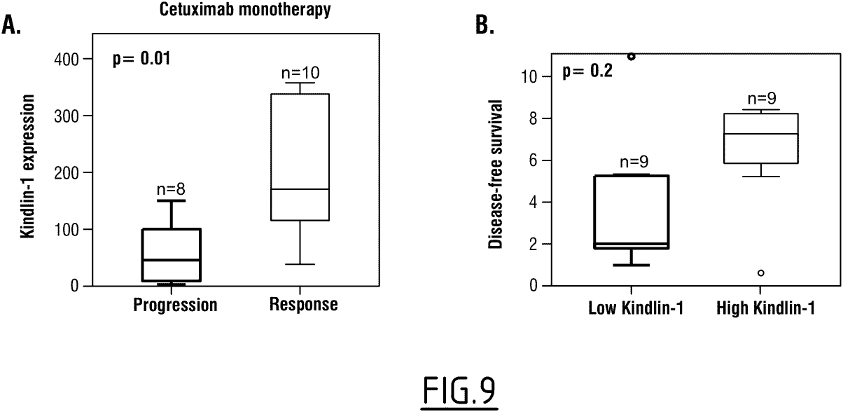

To further investigate the relevance of Kindlin-1 as a sensitive biomarker of EGFR/RAS pathway inhibitors response, a cohort of 18 head and neck cancer patients treated with Cetuximab monotherapy at the Curie Hospital was analyzed. We found that those patients with a good response to the treatment had higher Kindlin-1 expression levels than those that were not well responding, thus wherein the cancer progressed (FIG. 9A). Moreover, there was a tendency in patients with higher Kindlin-1 expression towards a delay in the progression of the disease (FIG. 9B).

### Example 2

### Materials and Methods

### Co-Immunoprecipitation

Cells were lysed using NP40 buffer (50 mM Tris-HCl, pH 7.5; 150 mM NaCl; 0.5% NP40) containing protease inhibitors (1:1000 orthovanadate, 1:1000 apoprotinine, 1:200 PMSF). Protein extracts were incubated with 1 mg antibodies for Kindlin-1, EGFR or normal rabbit IgG (GTX35035, GeneTex, Irvine, CA, USA) and 10 ml Sepharose Protein A beads (Rockland, Limerick, PA, USA) with rotation at 4° C. overnight. Beads were washed with NP40 buffer three times and immunoprecipitates were resolved by western blotting as previously described.

### Immunofluorescence

Cells were plated on fibronectin coated coverslips. After serum starvation overnight, cells were treated with 100 ng/ml EGF for the indicated time, fixed in 4% paraformaldehyde, permeabilized and immunostained with primary antibodies (anti-Kindlin-1, 1:700; anti-EGFR, 1:250, A-10, Santa Cruz Biotechnology, Santa Cruz, CA, USA) followed by alexa fluor-conjugated secondary antibodies (A11031 and A11034, Invitrogen, Carlsbad, CA, USA). Cells were then counterstained with DAPI and imaged with the fluorescence Eclipse Ti microscope from Nikon (Melville, NY, USA).

### MT Cell Viability Assay

15 000 cells were seeded in a 96-well plate and treated with Cetuximab (Erbitux 5 mg/ml, Merck Europe). After 48 and 72 hours cell viability was assessed using the CellTiter-Glo 2.0 Assay according to the instructions of the manufacturer (Promega, Madison, WI, USA). Luminiscence was recorded on a 96-well microplate reader (Promega Glomax Discover, Promega).

### Human Tumors

Raw data from different studies deposited in the GEO repository (http://www.ncbi.nlm.nih.gov/geo/) was also analyzed: 20 NSCL patients treated with Erlotinib (GEO accession number GSE33072), 40 HNSCC patients treated with Cetuximab (GEO accession number GSE65021) and 68 metastatic CRC patients treated with Cetuximab (GEO accession number GSE5851).

### Results

### Kindlin-1 Directly Interacts and Colocalizes With EGFR in Cancer Cells and Has an Impact on the Activation of EGFR Pathway

Due to the correlation found between Kindlin-1 and EGFR at mRNA and protein levels, it was hypothesized that these two proteins might interact in cancer cells. To address this question, the inventors chose BT20 triple negative breast cancer cells which present a high expression of Kindlin-1 and EGFR. Possible interaction at the endogenous level was examined by co-immunoprecipitation experiments. The inventors found that Kindlin-1 is able to immunoprecipitate EGFR and vice versa. Then, to further characterize this association, the subcellular localization of both proteins was examined by immunofluorescence. In absence of EGF stimulation, EGFR mainly localizes at the plasma membrane of BT20 cells while Kindlin-1 exhibits a dot-like staining predominantly at the perinuclear region. However, after 15 min EGF stimulation, promoting EGFR activation, EGFR is not anymore present at the cell surface. The receptor is internalized and colocalizes with Kindlin-1 in some ventral structures that should be further characterized.

As endogenous Kindlin-1 and EGFR proteins interact in cancer cells, the inventors wanted to study if Kindlin-1 could have an impact in the activation of the EGFR pathway. Therefore Kindlin-1 was depleted in different cancer cells lines (MDA-MB-468, H1975, BT20 and H358) and the activation of EGFR pathway checked using as a readout, the levels of phosphorylated ERK, one of the key effectors of the pathway. The inventors found that silencing Kindlin-1 drastically decreased the phosphorylation levels of ERK suggesting the involvement of Kindlin-1 in the EGFR pathway.

### Kindlin-1 Overexpression Renders Sensitive Cells More Resistant to Cetuximab

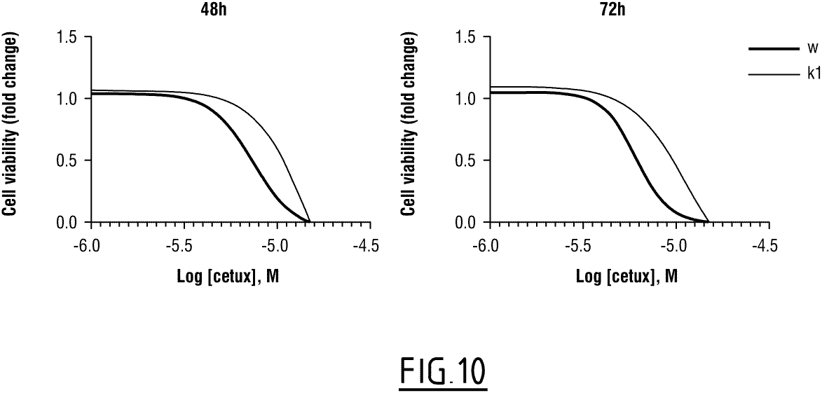

The possibility that Kindlin-1 ectopic expression could reverse the sensitivity to the EGFR inhibitor Cetuximab was tested. The inventors hypothesized that ectopic expression of Kindlin-1 could bypass the action of Cetuximab, maintaining the EGFR pathway activated and therefore rendering sensitive cells more resistant. To confirm our hypothesis, MDA-MB-468 cells stably overexpressing Kindlin-1 was generated (named k1). Control- and Kindlin-1-expressing MDA-MB-468 cells were treated with increasing concentrations of Cetuximab (1-20 µM) and a MT cell viability assay was performed after 48 h and 72 h of treatment. As expected, cells expressing ectopic Kindlin-1 became more resistant to the drug as determined by the calculation of the half maximal inhibitory concentration or IC50 (control cells: 7.3 µM (48 h); 6.1 µM (72 h); Kindlin-1 cells: 14.8 µM (48h); 11.3 µM (72 h)) ( see FIG. 10).

### Kindlin-1 Expression Predicts Cetuximab/Erlotinib Response in Head and Neck, Lung And Colorectal Cancer Patients

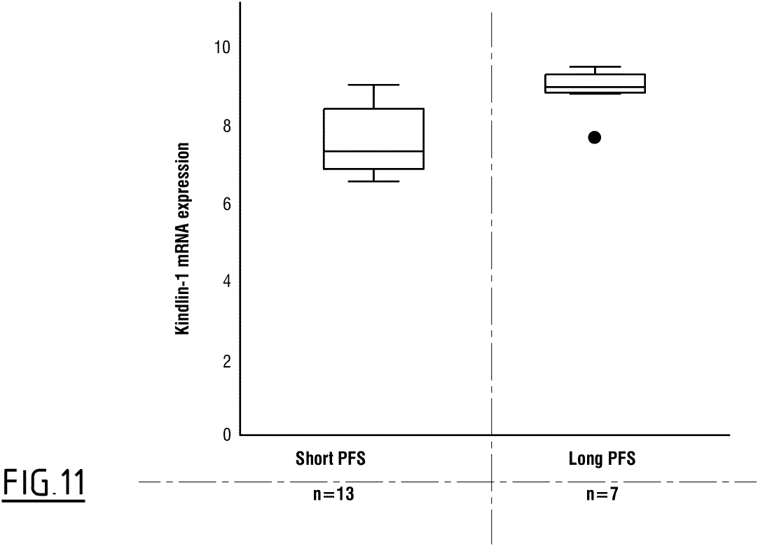

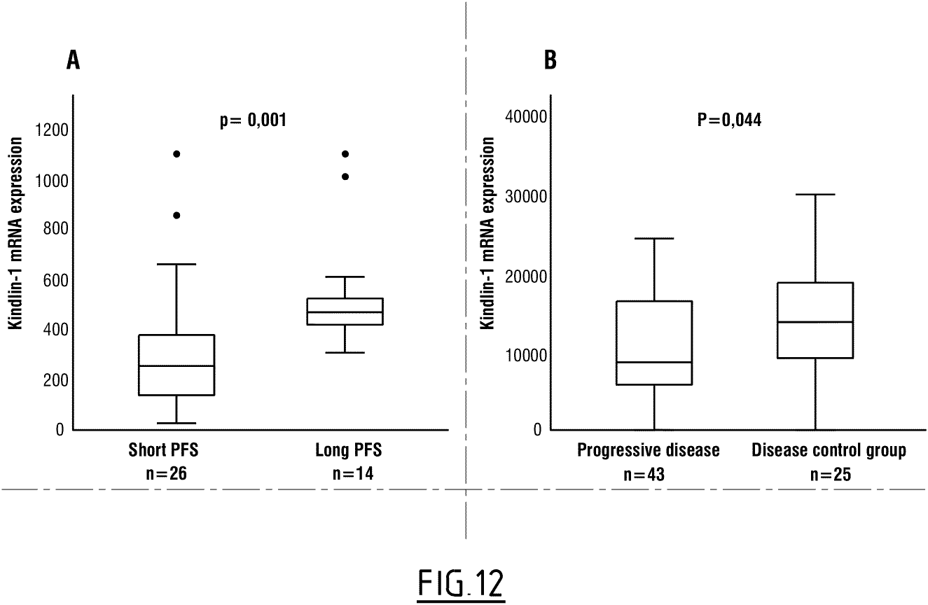

To further investigate the relevance of Kindlin-1 as a sensitive biomarker of the response to EGFR inhibitors, raw data from different studies deposited in the GEO repository were also studied: 20 NSCL patients treated with Erlotinib as well as 40 HNSCC and 68 metastatic CRC patients treated with Cetuximab were analysed. In all the three cohorts, a higher Kindlin-1 mRNA expression was found in patients with a better response and a longer patient relapse-free survival (FIGS. 11 and 12).

1. An ex vivo method for selecting a patient having cancer as eligible to EGFR/RAS pathway inhibitor therapy, comprising:
a) Measuring Kindlin-1 expression level in cancer cells of said patient having cancer;
b) Selecting the patient as eligible to EGFR/RAS pathway inhibitor therapy if the measured Kindlin-1 expression level in cancer cells of said patient having cancer is equal to, or above, a reference level.

2. The method according to claim 1, wherein:
i. the reference level is determined from the expression level of Kindlin-1 measured in samples of cancer cells sensitive to EGFR/RAS pathway inhibitor therapy, wherein said cancer cells sensitive to EGFR/RAS pathway inhibitor therapy are of the same type as the cancer cells of said patient having cancer, or
ii. the reference level is determined from the expression level of Kindlin-1 measured in samples of cancer cells resistant to EGFR/RAS pathway inhibitor therapy, wherein said cancer cells resistant to EGFR/RAS pathway inhibitor therapy are of the same type as the cancer cells of said patient having cancer, and the measured Kindlin-1 expression level in cancer cells of said patient having cancer is above the reference level.

3. The method according to any one of claims 1-2, wherein the cancer is a solid cancer, preferably an epithelial cancer.

4. The method according to any one of claims 1-3, wherein the cancer is breast, lung, colon, pancreas, bladder, or head and neck cancer, preferably a triple negative breast cancer.

5. The method according to any one of claims 1-4, wherein the EGFR/RAS pathway inhibitor therapy is an EGFR inhibitor, a MEK inhibitor, a BRAF inhibitor, or a RAS inhibitor therapy.

6. The method according to claim 5, wherein the EGFR inhibitor is selected from the group consisting of anti-EGFR antibodies, preferably Cetuximab, Panitumumab, Zalutumumab, Nimotuzumab, or Matuzumab; and small molecule inhibitors, preferably Erlotinib, Dabrafenib, Gefitinib, Osimertinib, Pelitinib, AZD3759, afatinib, brigatinib, icotinib, or lapatinib.

7. The method according to claim 5, wherein the MEK inhibitor is selected from the group consisting of PD0325901, CI-1040, Selumetinib, Trametinib, Cobimetinib, Refametinib and TAK-733.

8. The method according to claim 5, wherein the BRAF inhibitor is selected from the group consisting of vemurafenib, PLX-4720 and Dabrafenib.

9. The method according to claim 5, wherein the RAS inhibitor is AMG510.

10. The method according to claim 1, which further comprises administering an EGFR/RAS pathway inhibitor to said patient if identified as having a Kindlin-1 expression level in cancer cells equal to, or above, the reference level.

11. The method according to claim 10, wherein:
a. the reference level is determined from the expression level of Kindlin-1 measured in samples of cancer cells sensitive to EGFR/RAS pathway inhibitor therapy, wherein said cancer cells sensitive to EGFR/RAS pathway inhibitor therapy are of the same type as the cancer cells of said patient having cancer, or
b. the reference level is determined from the expression level of Kindlin-1 measured in samples of cancer cells resistant to EGFR/RAS pathway inhibitor therapy, wherein said cancer cells resistant to EGFR/RAS pathway inhibitor therapy are of the same type as the cancer cells of said patient having cancer.

12. The method according to claim 11, wherein the EGFR/RAS pathway inhibitor therapy is an EGFR inhibitor, a MEK inhibitor, a BRAF inhibitor, or a RAS inhibitor therapy.

13. A method for prognosis cancer outcome or progression in a patient suffering from EGFR/RAS-driven cancer, comprising:
a) Measuring Kindlin-1 expression level in cancer cells of said patient suffering from EGFR/RAS-driven cancer;
b) Determining that the cancer is likely to progress or have a poor outcome if the measured Kindlin-1 expression level in cancer cells of the patient is equal to, or above, a first reference level; or determining that the cancer is unlikely to progress or have a poor outcome if the measured Kindlin-1 expression level in cancer cells of the patient is below a second reference level.

14. The method for prognosis cancer outcome or progression according to claim 13, wherein the patient is suffering from a bladder or a head and neck cancer.

15. The method for prognosis cancer outcome or progression according to claim 13, wherein:
a. the first reference level is determined from the expression level of Kindlin-1 measured in samples of cancer cells of aggressive EGFR/RAS-driven cancers, and wherein said cancer cells are of the same type as the cancer cells of said patient having cancer, and
b. the second reference level is determined from the expression level of Kindlin-1 measured in samples of cancer cells of non-aggressive EGFR/RAS-driven cancers, and wherein said cancer cells are of the same type as the cancer cells of said patient having cancer.

16. The method for prognosis cancer outcome or progression according to claim 13, which further comprises administering an EGFR/RAS pathway inhibitor or a Kindlin-1 inhibitor to said patient if identified as having a cancer likely to progress or having a poor outcome.
